(19) United States
(12) Patent Application Publication
(10) Pub. No.: US 2019/0028810 A1
(43) Pub. Date: Jan. 24, 2019

(54) ODOR-SPREADING SPEAKER DEVICE

(71) Applicant: Jetvox Acoustic Corp., Taoyuan (TW)

(72) Inventor: To-Teng HUANG, Taoyuan (TW)

(21) Appl. No.: 15/724,644

(22) Filed: Oct. 4, 2017

(30) Foreign Application Priority Data
Jul. 21, 2017 (TW) 106210806

(51) Int. Cl. H04R 9/04; H04R 7/02; A61L 9/012

(52) U.S. Cl. CPC H04R 9/046; A61L 2209/15; A61L 9/012; H04R 7/02

(57) ABSTRACT

An odor-spreading speaker device includes a voice coil assembly and a fragrant sheet for spreading a certain odor. The voice coil assembly at least includes a vibration diaphragm, a separation ring, a magnet member, a yoke, a vibration coil, and a circuit board. The vibration diaphragm, the separation ring, and the magnet member are sequentially stacked with one another. The yoke at least surrounds the magnet member. The vibration coil assembly is disposed between the magnet member and the yoke, and the vibration coil assembly surrounds the magnet member. The circuit board is electrically connected to the vibration coil assembly. The fragrant sheet is fixed to the voice coil assembly directly or indirectly. Accordingly, when the voice coil assembly is operated to make sounds, the sound vibration drives airflows to pass through the fragrant sheet, so that the odor-spreading effect can be enhanced.

### CROSS-REFERENCE TO RELATED APPLICATION

This non-provisional application claims priority under 35 U.S.C. § 119(a) to Patent Application No. 106210806 filed in Taiwan, R.O.C. on Jul. 21, 2017, the entire contents of which are hereby incorporated by reference.

### BACKGROUND

### Technical Field

The instant disclosure relates to a speaker device, in particular, to an odor-spreading speaker device.

### Related Art

3C (computer, communication, and consumer electronics) devices with multiple sensual enjoyments are gradually developed. For instance, a device may be adapted to provide audio-visual enjoyments as well as body sensations for the users. For another instance, a device may be adapted to provide audio-visual enjoyments as well as fragrant smell enjoyments for the users at the same time.

A conventional speaker device or earphone with fragrance may be divided into two types. A common approach is just to attach a perfume-absorbed material to the speaker device simply, so that the speaker device can continuously spread the fragrance. The intensity of the odor depends on the residual concentration of the fragrant material, and the intensity of the fragrant odor cannot be changed along with the operation of the speaker device. The other approach is to assemble a small fan in the speaker device. During the operation of the speaker device, the fan blows winds outwardly to enhance the intensity of the fragrant odor. However, the volume of the speaker device has to be enlarged when the fan is assembled in the speaker device. As a result, such approach is not suitable for earphones. Furthermore, the fans would lead further electricity consumption. Therefore, to portable devices with limited electricity, the speaker device with the fan would have shorter operation time as compared with one without the fan. Additionally, the fan is also a noise source and an electromagnetic interference source which may adversely affect the audio performance of the speaker device.

### SUMMARY

According to the instant disclosure, an odor-spreading speaker device is provided. Accordingly, during the operation of the speaker device, the odor-spreading function can be achieved without additional airflow driving devices.

In view of this, an odor-spreading speaker device is provided. In one embodiment, the speaker device comprises a voice coil assembly and a fragrant sheet for spreading a certain odor. The voice coil assembly at least comprises a vibration diaphragm, a separation ring, a magnet member, a yoke, a vibration coil, and a circuit board. The vibration diaphragm, the separation ring, and the magnet member are sequentially stacked with one another. The yoke at least surrounds the magnet member.

The vibration coil assembly is disposed between the magnet member and the yoke, and the vibration coil assembly surrounds the magnet member. The circuit board is electrically connected to the vibration coil assembly. The fragrant sheet is fixed to the voice coil assembly directly or indirectly. Accordingly, when the voice coil assembly is operated to make sounds, the sound vibration drives airflows to pass through the fragrant sheet, so that the odor-spreading effect can be enhanced.

In one embodiment, the yoke is a U-shaped yoke. The yoke receives the vibration diaphragm, the separation ring, and the magnet member. A periphery and a bottom of the magnet member are surrounded by the yoke.

In one embedment, the odor-spreading speaker device further comprises a front housing and a rear housing. The front housing at least has a main sound output opening defined through an inner side of the front housing and an outer side of the front housing. The rear housing is combined with the front housing to form a protecting space. The rear housing has a rear opening. The yoke of the voice coil assembly is engaged in the rear opening, and the vibration diaphragm is located in the protecting space. The fragrant sheet is fixed to the front housing or the rear housing.

In one embodiment, the fragrant sheet is disposed on an inner lateral surface of the front housing and covers the main audio output opening.

In one embodiment, the odor-spreading speaker device further comprises a rear audio output opening defined through the separation ring, the magnet member, and the yoke. The fragrant sheet is disposed on a protruding portion of the yoke and the fragrant sheet covers the rear audio output opening.

In one embodiment, the front housing further comprises a plurality of auxiliary audio output openings. The auxiliary audio output openings surround the main audio output opening, and the auxiliary audio output openings are defined through the inner side of the front housing and the outer side of the front housing. The fragrant sheet at least covers one of the auxiliary audio output openings.

In one embodiment, the rear housing further comprises at least one auxiliary opening, and the fragrant sheet covers the at least one auxiliary opening.

In one embodiment, the fragrant sheet is integrally formed with the front housing or with the rear housing as a whole.

In one embodiment, the fragrant sheet is combined with the vibration diaphragm.

In one embodiment, the fragrant sheet is disposed in the voice coil assembly and stacked with the vibration diaphragm, the separation ring, and the magnet member.

According to some embodiments of the instant disclosure, the fragrant sheet is combined with the speaker device. Therefore, the speaker device may be an individual component with the odor-spreading function, and the speaker device can be further assembled to a casing of an earphone or a loudspeaker. During the operation of the speaker device, the sound vibration drives airflow, so that the odor-spreading effect becomes more apparent. As a result, the speaker device can be adapted to create a certain scenario with the certain odor without assembling additional airflow generating devices.

### BRIEF DESCRIPTION OF THE DRAWINGS

The disclosure will become more fully understood from the detailed description given herein below for illustration only, and thus not limitative of the disclosure, wherein:

FIG. 1 illustrates an exploded view of an odor-spreading speaker device according to a first embodiment of the instant disclosure;

FIG. 2 illustrates a perspective view of the odor-spreading speaker device of the first embodiment;

FIG. 3 illustrates a cross-sectional view of the odor-spreading speaker device of the first embodiment;

FIG. 4 illustrates an exploded view of an odor-spreading speaker device according to a second embodiment of the instant disclosure;

FIG. 5 illustrates a cross-sectional view of an odor-spreading speaker device according to a third embodiment of the instant disclosure;

FIG. 6 illustrates a cross-sectional view of an odor-spreading speaker device according to a fourth embodiment of the instant disclosure;

FIGS. 7 and 8 illustrate exploded views of an odor-spreading speaker device according to a fifth embodiment of the instant disclosure;

FIG. 9 illustrates an exploded view of an odor-spreading speaker device according to a sixth embodiment of the instant disclosure; and

FIG. 10 illustrates an exploded view of an odor-spreading speaker device according to a seventh embodiment of the instant disclosure.

### DETAILED DESCRIPTION

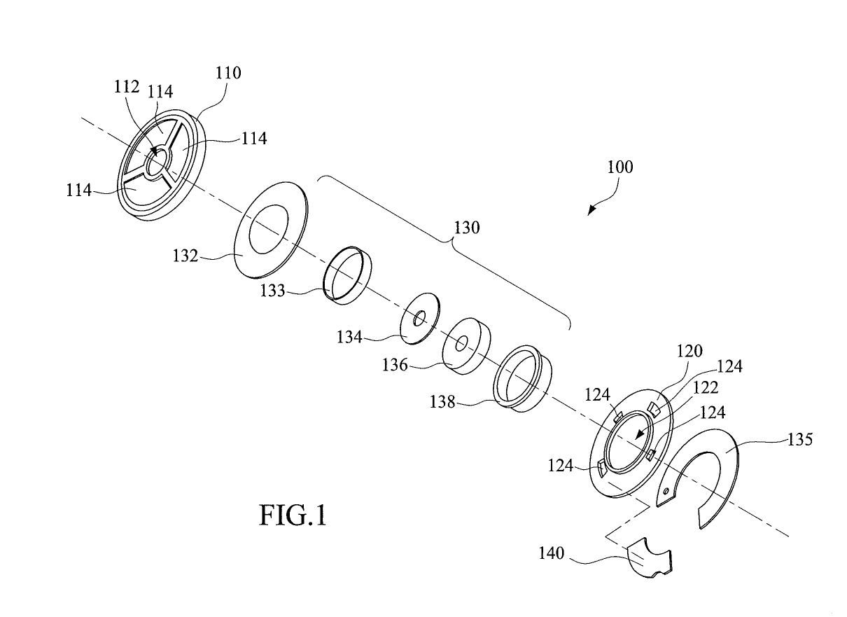

Please refer to FIGS. 1 to 3, illustrating an odor-spreading speaker device 100 according to a first embodiment of the instant disclosure. The odor-spreading speaker device 100 is adapted to spread a certain fragrance when it is operated. In the first embodiment, the odor-spreading speaker device 100 comprises a front housing 110, a rear housing 120, a voice coil assembly 130, and a fragrant sheet 140.

As shown in FIGS. 1 to 3, the front housing 110 at least has a main audio output opening 112 and a plurality of auxiliary audio output openings 114. The main audio output opening 112 is approximately located at a middle portion of the front housing 110, and the main audio output opening 112 is defined through an inner side of the front housing 110 and an outer side of the front housing 110. The auxiliary audio output openings 114 surround the main audio output opening 112, and the auxiliary audio output openings 114 are defined through the inner side of the front housing 110 and the outer side of the front housing 110.

As shown in FIGS. 1 to 3, the rear housing 120 is combined with the front housing 110 to form a protecting space. The rear housing 120 has a rear opening 122 and an auxiliary opening 124. The rear opening 122 is approximately located at a middle portion of the rear housing 120 for engaging with the voice coil assembly 130. The fragrant sheet 140 may be disposed on an outer side of the rear housing 120, so that the fragrant sheet 140 is indirectly fixed to the voice coil assembly 130.

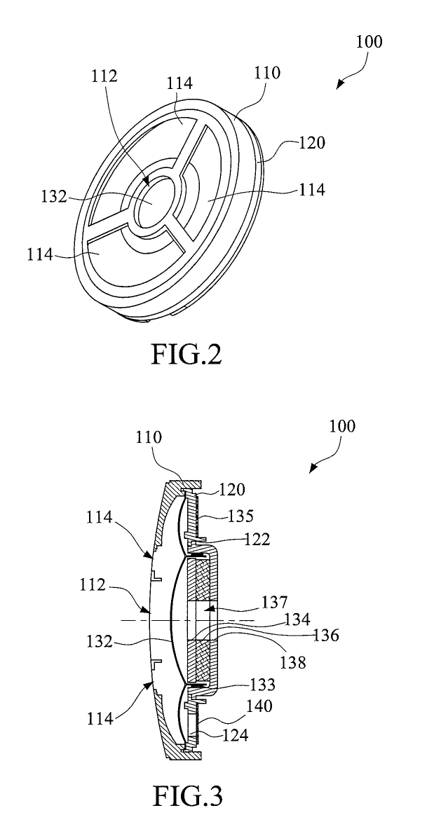

As shown in FIGS. 1 and 3, the voice coil assembly 130 is engaged in the rear opening 122 of the rear housing 120, so that the front housing 110, the rear housing 120, and the voice coil assembly 130 are combined with each other to form a monomer (i.e., the speaker device 100 shown in FIG. 2). Therefore, the speaker device 100 may be further combined with an earphone housing or a loudspeaker housing to form an earphone or a loudspeaker, respectively.

As shown in FIGS. 1 and 3, the voice coil assembly 130 at least comprises a vibration diaphragm 132, a separation ring 134, a magnet member 136, a yoke 138, a vibration coil 133, and a circuit board 135. The vibration diaphragm 132, the separation ring 134, and the magnet member 136 are stacked with one another in order. The yoke 138 at least surrounds the magnet member 136. In one embodiment, the yoke 138 is a U-shaped yoke to receive the vibration diaphragm 132, the separation ring 134, and the magnet member 136, so that a periphery and a bottom of the magnet member 136 are surrounded by the yoke 138. Moreover, the yoke 138 is engaged into the rear opening 122 of the rear housing 120 and protruding outwardly. The vibration diaphragm 132 is located in the protecting space. The fragrant sheet 140 is fixed to the front housing 110 or the rear housing 120. The vibration coil 133 is disposed between the magnet member 136 and the yoke 138 and surrounds the magnet member 136. The circuit board 135 is electrically connected to the vibration coil 133 for supplying electricity to the vibration coil 133, so that the vibration coil 133 is driven to generate an essential electromagnetic-field change and to drive the vibration diaphragm 132 to make sounds. As long as the circuit board 135 is electrically connected to the vibration coil 133, the circuit board 135 may be fixed inside or outside the yoke 138, or the circuit board 135 may be fixed to another external component (e.g., the rear housing 120). Furthermore, the voice coil assembly 130 further comprises a rear audio output opening 137 defined through the separation ring 134, the magnet member 136, and the yoke 138. The configuration and modified configurations of the assembly of the vibration diaphragm 132, the separation ring 134, the magnet member 136, the yoke 138, the vibration coil 133, and the circuit board 135 are not mentioned again in the following paragraphs.

The fragrant sheet 140 is used to spread a certain odor. Materials with the certain odor are attached onto the fragrant sheet 140 via coating or adhering manners. For example, liquids with certain odors may be coated onto the surface of the fragrant sheet 140 by spraying, immersion, dipping, etc., and the liquids are further adhered to the interior of the fragrant sheet 140. The fragrant sheet 140 may be made from multiporous materials or fiber materials to adhere liquids with certain odors, e.g., perfumes, essential oils, or other liquids with certain odors. Hence, the fragrant sheet 140 has to possess great adhesive abilities, and the material for making the fragrant sheet 140 is not limited. The material for making the fragrant sheet 140 may be plastics (especially mulitporous plastics), mesh fabrics, metals (metal meshes, metal wools, and multiporous sintered metal sheets), fibers, or so forth. It is also understood that the source of the odor of the fragrant sheet 140 is not limited to the externally-added perfumes, essential oils, or liquids with certain odors. During the manufacturing of the fragrant sheet 140, liquids or solids with certain odors may be mixed with the materials for making the fragrant sheet 140. The fragrant sheet 140 is fixed to the voice coil assembly 130 directly or indirectly. In the first embodiment, the fragrant sheet 140 is indirectly fixed to the voice coil assembly 130 via the front housing 110 or the rear housing 120. Specifically, in the embodiment, the fragrant sheet 140 is directly fixed on an outer lateral surface of the rear housing 120 and the fragrant sheet 140 covers the auxiliary opening 124, so that the fragrant sheet 140 is indirectly fixed to the voice coil assembly 130. It is understood that the configuration of the fragrant sheet 140 is not limited to the aforementioned embodiments.

In some embodiments, the fragrant sheet 140 is fixed to the voice coil assembly 130 directly or indirectly. When the vibration coil 133 of the voice coil assembly 130 receives the electricity signals of the circuit board 135, the vibration diaphragm 132 is driven to make sounds. The sound vibration also drives airflows. Hence, the airflows pass through the fragrant sheet 140 and take away molecules with certain odors. Therefore, during the operation of the speaker device 100, when the sounds are made, certain odors may be also spread by the speaker device 100. As a result, the speaker device 100 may be adapted to create a certain scenario with the certain odor.

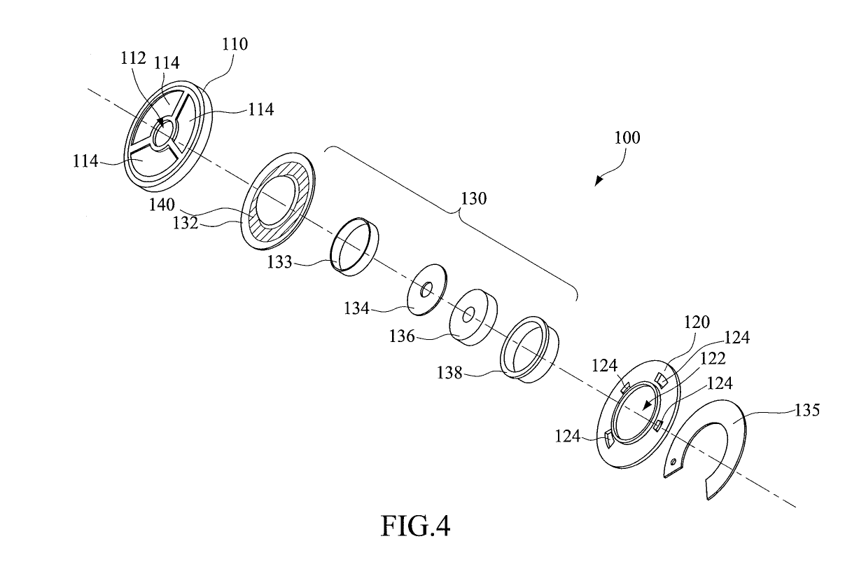

Please refer to FIG. 4, illustrating an odor-spreading speaker device 100 according to a second embodiment of the instant disclosure. The fragrant sheet 140 may be combined with the vibration diaphragm 132 to become a part of the vibration diaphragm 132. For example, the fragrant sheet 140 may be a fiber layer located on the surface of the vibration diaphragm 132. Therefore, when the vibration diaphragm 132 is driven to vibrate, the fragrant sheet 140 is also vibrated, so that the odor-spreading effect would be more apparent. It is understood that the circular configured fragrant sheet 140 shown in the figure is provided as an illustrative purpose, and the distribution of the fragrant sheet 140 over the vibration diaphragm 132 is not limited thereto.

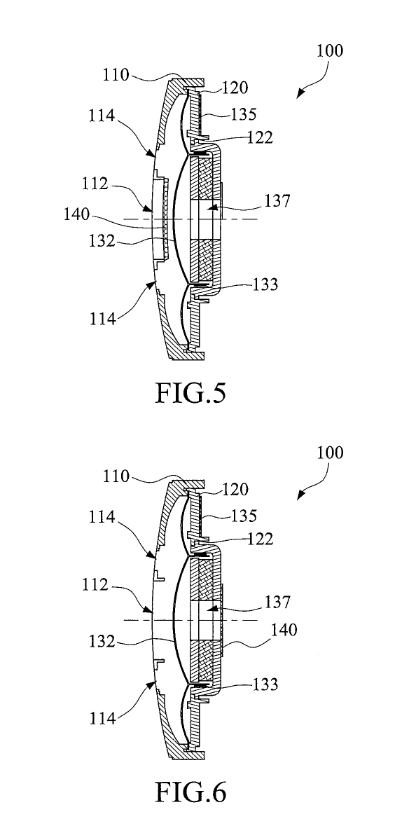

Please refer to FIG. 5, illustrating an odor-spreading speaker device 100 according to a third embodiment of the instant disclosure. In the third embodiment, the fragrant sheet 140 is disposed on an inner surface of the front housing 110 and the fragrant sheet 140 covers the main audio output opening 112. In this embodiment, the fragrant sheet 140 is also provided as a sound tuning member for adjusting the sound output of the speaker device 100. It is understood that the speaker device 100 may further have other sound tuning members assembled on other portions of the speaker device 100.

Please refer to FIG. 6, illustrating an odor-spreading speaker device 100 according to a fourth embodiment of the instant disclosure. In the fourth embodiment, the fragrant sheet 140 is disposed on a protruding portion of the yoke 138 and the fragrant sheet 140 covers the rear audio output opening 137. Accordingly, the fragrant sheet 140 is also provided as a filtering member for preventing undesired particles entering into the speaker device 100 via the rear housing 120. In this embodiment, the fragrant sheet 140 may be a protecting net, a filament paper, a breathable foam (sponge), etc.

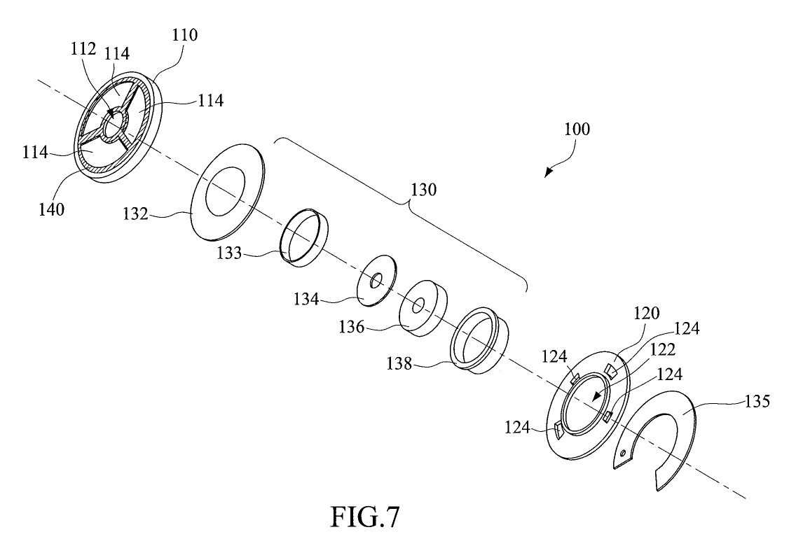

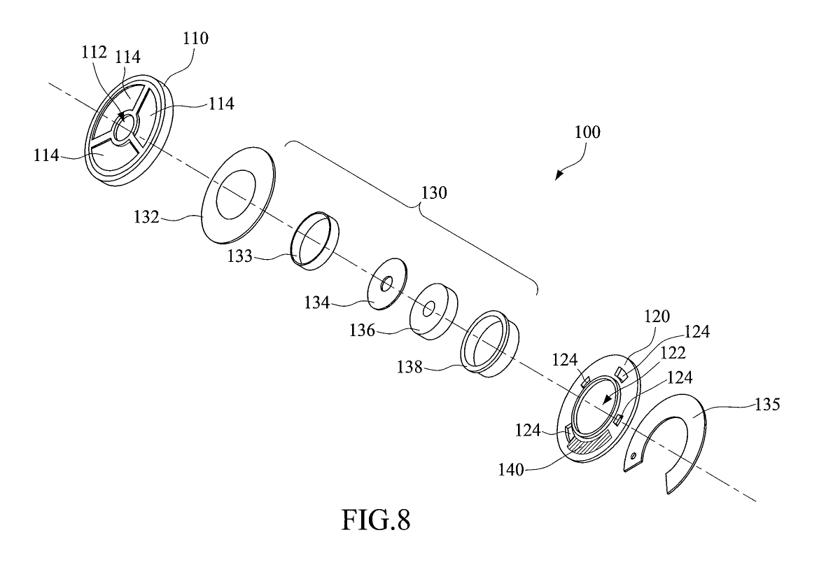

Please refer to FIGS. 7 and 8, illustrating an odor-spreading speaker device 100 according to a fifth embodiment of the instant disclosure. In the fifth embodiment, the front housing 110 and the rear housing 120 are made of plastic materials. Upon the manufacturing of the front housing 110 and the rear housing 120, the fragrant sheet 140 is processed to be particles in advance, and then the particles are mixed with the front housing 110 or the rear housing 120. In another embodiment, the fragrant sheet 140 may be combined with the surface of the front housing 110 or the surface of the rear housing 120 via a hetero-type injection molding technique. Hence, the fragrant sheet 140 can be integrally formed with the front housing 110 or with the rear housing 120 as a whole. It is understood that, the distribution of the fragrant sheet 140 shown in the figure is provided for illustrative purpose, and the distribution of the fragrant sheet 140 over the front housing 110 and the rear housing 120 is not limited thereto.

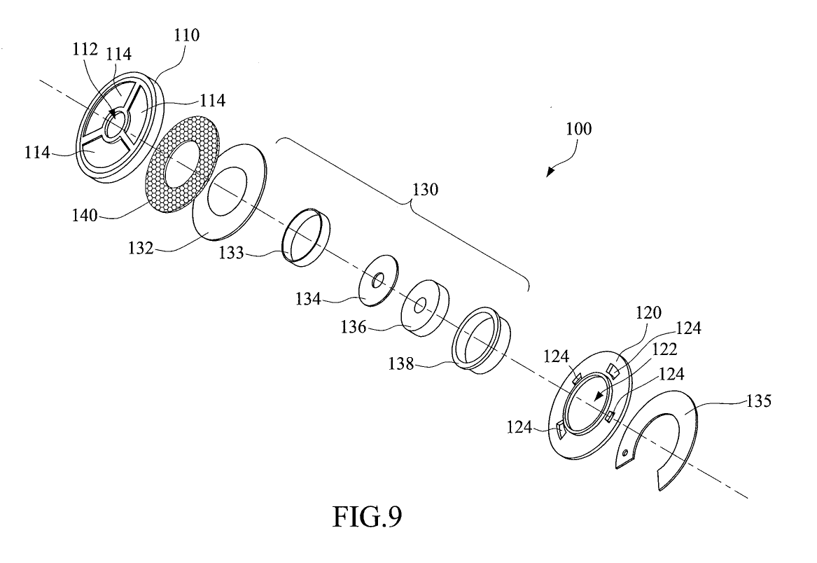

Please refer to FIG. 9, illustrating an odor-spreading speaker device 100 according to a sixth embodiment of the instant disclosure. In the sixth embodiment, the fragrant sheet 140 is disposed on an inner lateral surface of the front housing 110 and the fragrant sheet 140 at least covers one of the auxiliary audio output opening 114.

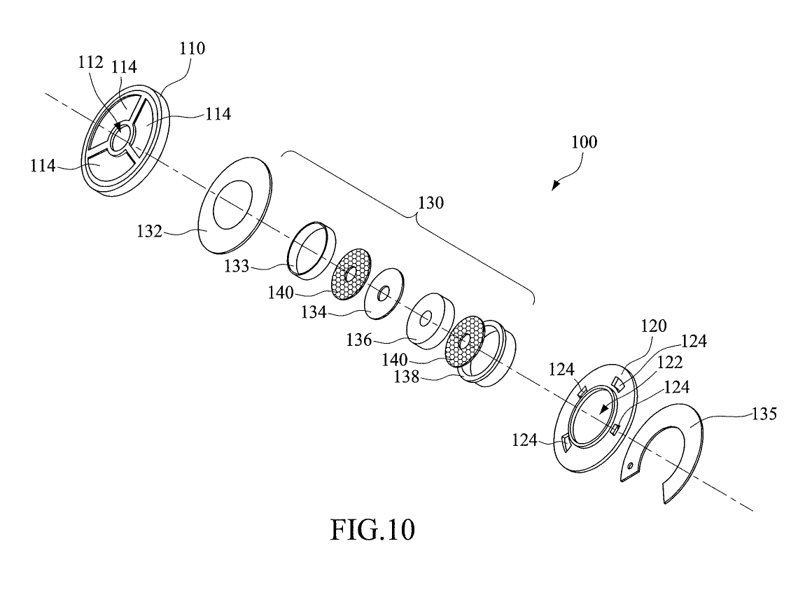

Please refer to FIG. 10, illustrating an odor-spreading speaker device 100 according to a seventh embodiment of the instant disclosure. In the seventh embodiment, the fragrant sheet 140 is disposed in the voice coil assembly 130, and the number of the fragrant sheet 140 may be more than one. The fragrant sheet 140 is stacked with the vibration diaphragm 132, the separation ring 134, and the magnet member 136, so that the fragrant sheet 140 is integrated in the voice coil assembly 130.

According to some embodiments of the instant disclosure, the fragrant sheet 140 is combined with the speaker device 100. Hence, the fragrant sheet 140 becomes a part of the speaker device 100. The fragrant sheet 140 may be an additional component used for adhering odor materials; the fragrant sheet 140 may be a direct replacement for a sound tuning member or a replacement for a filtering member or may be integrated with a certain component (e.g., the vibration diaphragm 132, the front housing 110, and the rear housing 120). Therefore, the speaker device 100 may be an individual component with the odor-spreading function, and the speaker device 100 can be further assembled to a casing of an earphone or a loudspeaker. During the operation of the speaker device 100, the sound vibration drives airflow, so that the odor-spreading effect becomes more apparent. As a result, the speaker device 100 can be adapted to create a certain scenario with the certain odor.

What is claimed is:

1. An odor-spreading speaker device, comprising:
a voice coil assembly, at least comprising a vibration diaphragm, a separation ring, a magnet member, a yoke, a vibration coil, and a circuit board, wherein the vibration diaphragm, the separation ring, and the magnet member are sequentially stacked with one another, the yoke at least surrounds the magnet member, the vibration coil is disposed between the magnet member and the yoke, and the vibration coil surrounds the magnet member, and the circuit board is electrically connected to the vibration coil; and
a fragrant sheet spreading a certain odor, wherein the fragrant sheet is directly or indirectly fixed to the voice coil assembly.

2. The odor-spreading speaker device according to claim 1, wherein the yoke is a U-shaped yoke, the yoke receives the vibration diaphragm, the separation ring, and the magnet member, and a periphery and a bottom of the magnet member are surrounded by the yoke.

3. The odor-spreading speaker device according to claim 1, further comprising:
a front housing, at least having a main audio output opening, wherein the main audio output opening is defined through an inner side of the front housing and an outer side of the front housing; and
a rear housing combined with the front housing to form a protecting space, wherein the rear housing has a rear opening, the yoke is engaged in the rear opening, and the vibration diaphragm is located in the protecting space, and the fragrant sheet is fixed to the front housing or the rear housing.

4. The odor-spreading speaker device according to claim 3, wherein the fragrant sheet is disposed on an inner lateral surface of the front housing and the fragrant sheet covers the main audio output opening.

5. The odor-spreading speaker device according to claim 3, further comprising a rear audio output opening, defined through the separation ring, the magnet member, and the yoke, wherein the fragrant sheet is disposed on a protruding portion of the yoke and the fragrant sheet covers the rear audio output opening.

6. The odor-spreading speaker device according to claim 3, wherein the front housing further comprises a plurality of auxiliary audio output openings, the auxiliary audio output openings surround the main audio output opening and are defined through the inner side of the front housing and the outer side of the front housing, the fragrant sheet at least covers one of the auxiliary audio output openings.

7. The odor-spreading speaker device according to claim 3, wherein the rear housing further comprises at least one auxiliary opening, and the fragrant sheet covers the at least one auxiliary opening.

8. The odor-spreading speaker device according to claim 3, wherein the fragrant sheet is integrally formed with the front housing or with the rear housing as a whole.

9. The odor-spreading speaker device according to claim 1, wherein the fragrant sheet is combined with the vibration diaphragm.

10. The odor-spreading speaker device according to claim 1, wherein the fragrant sheet is disposed in the voice coil assembly and stacked with the vibration diaphragm, the separation member, and the magnet member.
